(19) United States
(12) Patent Application Publication
(10) Pub. No.: US 2026/0076640 A1
(43) Pub. Date: Mar. 19, 2026

(54) ULTRASONIC DEVICES CONFIGURED FOR BLOOD PRESSURE ESTIMATION

(71) Applicant: QUALCOMM Incorporated, San Diego, CA (US)

(72) Inventors: Sumit AGRAWAL, Sunnyvale, CA (US); Hrishikesh Vijaykumar PANCHAWAGH, Cupertino, CA (US); Emily Kathryn BROOKS, Amherst, NY (US); Kostadin Dimitrov DJORDJEV, Los Gatos, CA (US); John Keith SCHNEIDER, Williamsville, NY (US)

(21) Appl. No.: 18/885,358

(22) Filed: Sep. 13, 2024

(51) Int. Cl. A61B 8/04; A61B 5/00; A61B 5/021; A61B 5/026; A61B 5/05; A61B 8/00; A61B 8/02; A61B 8/06; A61B 8/08

(52) U.S. Cl. CPC A61B 8/04; A61B 5/02141; A61B 5/026; A61B 5/0261; A61B 5/05; A61B 5/7275; A61B 8/02; A61B 8/06; A61B 8/085; A61B 8/4227; A61B 8/4427; A61B 8/4494; A61B 8/488; A61B 8/5223; A61B 8/54

(57) ABSTRACT

Some disclosed blood pressure estimation methods involve controlling, by a control system, an ultrasonic sensor system to transmit ultrasonic waves to a target object on an outer surface of an apparatus, the ultrasonic sensor system having M ultrasonic receiver elements in a single array. Some such methods involve receiving, by the control system from the ultrasonic sensor system, ultrasonic receiver signals from each of a plurality of the M ultrasonic receiver elements in the single array, the ultrasonic receiver signals corresponding to ultrasonic waves reflected from the target object. Some such methods involve detecting, by the control system, an artery within the target object based on the ultrasonic receiver signals. Some such methods involve estimating, by the control system, a blood pressure within the artery based on the ultrasonic receiver signals.

### TECHNICAL FIELD

This disclosure relates to ultrasonic devices and more specifically to estimating blood pressure based, at least in part, in data obtained via ultrasonic devices.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive and ambulatory monitoring. Some such devices are, or include, ultrasonic devices. Although some ultrasonic-enabled devices and systems have previously been deployed, improved devices and systems would be desirable.

### SUMMARY

The systems, methods and devices of this disclosure each have several aspects, no single one of which is solely responsible for the desirable attributes disclosed herein.

One innovative aspect of the subject matter described in this disclosure can be implemented in an apparatus. The apparatus may include an ultrasonic sensor system and a control system. The ultrasonic sensor system may be, or may include, a single array of M ultrasonic receiver elements. M may be an integer of 2 or more. The single array may, in some examples, be a linear array. In some implementations, a mobile device (such as a wearable device, a hand-held device, etc.) may be, or may include, at least part of the apparatus. However, according to some implementations, the apparatus may be configured to be a hand-held device.

The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system may be configured to control the ultrasonic sensor system to transmit ultrasonic waves to a target object on an outer surface of the apparatus. The control system may be configured to receive, from the ultrasonic sensor system, ultrasonic receiver signals from each of a plurality of the M ultrasonic receiver elements in the single array. The ultrasonic receiver signals may correspond to ultrasonic waves reflected from the target object.

The control system may be configured to detect an artery within the target object based, at least in part, on the ultrasonic receiver signals. The control system may be configured to estimate a blood pressure within the artery based, at least in part, on the ultrasonic receiver signals.

In some examples, the artery may be a radial artery. However, in other examples the artery may be another type of artery.

According to some examples, the control system may be configured to apply a receiver-side beamforming process to the ultrasonic receiver signals, to produce a beamformed ultrasonic receiver image.

In some examples, the control system may be configured to estimate a change in cross-sectional area of the artery based at least in part on the beamformed ultrasonic receiver image. Estimating the blood pressure may be based in part on the change in the cross-sectional area of the artery.

According to some examples, the control system may be further configured to estimate a change in a blood flow rate within the artery based on the ultrasonic receiver signals. Estimating the blood pressure may be based in part on the change in the blood flow rate within the artery and the change in cross-sectional area of the artery.

In some examples, estimating the blood pressure may be based in part on a derivative of the blood flow rate (Q) within the artery with respect to the cross-sectional area (A) of the artery (dQ/dA). In some such examples, the control system may be configured to estimate a pulse wave velocity based on the derivative dQ/dA.

According to some examples, the control system may be configured to estimate the change in the blood flow rate within the artery based at least in part on speckle decorrelation-based blood flow measurements or based at least in part on a Doppler-based method.

In some examples, the apparatus may include a magnetic sensor system. In some such examples, the control system may be further configured to estimate a change in a blood flow rate within the artery based on magnetic sensor signals from the magnetic sensor system. In some such examples, estimating the blood pressure may be based in part on the change in the blood flow rate within the artery and the change in cross-sectional area of the artery.

According to some examples, the apparatus may include an optical sensor system. In some such examples, the control system may be further configured to estimate a change in a blood flow rate within the artery based on optical sensor signals from the optical sensor system. According to some such examples, estimating the blood pressure may be based in part on the change in the blood flow rate within the artery and the change in cross-sectional area of the artery.

In some examples, the ultrasonic sensor system may be configured to provide ultrasonic receiver signals to the control system at a frame rate in a range from 1 KHz to 3 KHz. According to some examples, the ultrasonic receiver signals include frequencies in a range from 10 MHz to 25 MHz. In some examples, the single array of M ultrasonic receiver elements may be linearly arranged.

Other innovative aspects of the subject matter described in this disclosure can be implemented in one or more methods. Some methods may involve controlling, by a control system, an ultrasonic sensor system to transmit ultrasonic waves to a target object on an outer surface of an apparatus. The ultrasonic sensor system may have M ultrasonic receiver elements in a single array.

Some methods may involve receiving, by the control system from the ultrasonic sensor system, ultrasonic receiver signals from each of a plurality of the M ultrasonic receiver elements in the single array, the ultrasonic receiver signals corresponding to ultrasonic waves reflected from the target object. Some methods may involve detecting, by the control system, an artery within the target object based, at least in part, on the ultrasonic receiver signals. Some methods may involve estimating, by the control system, a blood pressure within the artery based, at least in part, on the ultrasonic receiver signals.

Some methods may involve estimating, by the control system, a change in cross-sectional area of the artery based at least in part the ultrasonic receiver signals. Estimating the blood pressure may be based in part on the change in the cross-sectional area of the artery.

Some methods may involve estimating, by the control system, a change in a blood flow rate within the artery based on the ultrasonic receiver signals. Estimating the blood pressure may be based in part on the change in the blood flow rate within the artery.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in one or more non-transitory media having software stored thereon. The software may include instructions for controlling one or more devices to perform one or more disclosed methods. Some methods may involve controlling, by a control system, an ultrasonic sensor system to transmit ultrasonic waves to a target object on an outer surface of an apparatus. The ultrasonic sensor system may have M ultrasonic receiver elements in a single array.

Some methods may involve receiving, by the control system from the ultrasonic sensor system, ultrasonic receiver signals from each of a plurality of the M ultrasonic receiver elements in the single array, the ultrasonic receiver signals corresponding to ultrasonic waves reflected from the target object. Some methods may involve detecting, by the control system, an artery within the target object based, at least in part, on the ultrasonic receiver signals. Some methods may involve estimating, by the control system, a blood pressure within the artery based, at least in part, on the ultrasonic receiver signals.

Some methods may involve estimating, by the control system, a change in cross-sectional area of the artery based at least in part the ultrasonic receiver signals. Estimating the blood pressure may be based in part on the change in the cross-sectional area of the artery.

Some methods may involve estimating, by the control system, a change in a blood flow rate within the artery based on the ultrasonic receiver signals. Estimating the blood pressure may be based in part on the change in the blood flow rate within the artery.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 is a block diagram that shows example components of an apparatus according to some disclosed implementations.

FIG. 2 shows an example of a previously-deployed ultrasonic-based device for BP estimation.

FIG. 3 shows an example of a ultrasonic-based device for BP estimation according to the present disclosure.

FIG. 4A show examples of an apparatus that includes an ultrasonic sensor system.

FIG. 4B shows a top view of the ultrasonic sensor system 102 of FIG. 4A.

FIG. 5 shows example components of an apparatus according to some disclosed implementations.

FIG. 6 shows an example of an apparatus that is configured to perform a receiver-side beamforming process.

FIG. 7 is a flow diagram that shows examples of some disclosed operations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

Non-invasive health monitoring devices, such as ultrasonic-based devices and photoacoustic plethysmography (PAPG)-capable devices, have various potential advantages over more invasive health monitoring devices such as cuff-based or catheter-based blood pressure (BP) measurement devices. However, it has proven to be difficult to design satisfactory ultrasonic-based and PAPG-based devices for BP estimation. Some previously-deployed PAPG-based devices for BP estimation include two spatially separated ultrasonic receivers for measuring pulse wave velocity (PWV) and a separate PAPG sensor element for measuring the cross-sectional area of an artery. However, providing both ultrasonic receivers in a single device with, for example, a smart watch form factor resulted in a limited ultrasonic receiver separation and error-prone PWV estimations. Providing a single device that had ultrasonic receivers spaced far enough apart for reasonably accurate PWV measurements required a large form factor, which was not acceptable for some consumers. Moreover, having a PAPG sensor element and 2 ultrasonic receivers in a single device leads to a somewhat cumbersome arrangement: for example, it is challenging to align all three sensor elements to a single artery in order to obtain accurate measurements of arterial diameter, arterial distension and PWV.

Some disclosed devices are configured to estimate both PWV and the cross-sectional area of an artery based on measurements taken at a single arterial location. Some such devices include an ultrasonic sensor system and a control system. The ultrasonic sensor system may be configured to transmit ultrasonic waves and to receive ultrasonic waves reflected by a target object. According to some implementations, the ultrasonic sensor system may include a single array of ultrasonic receiver elements. In some implementations, the control system may be configured to receive ultrasonic receiver signals from each of the ultrasonic receiver elements in the single array. The ultrasonic receiver signals may correspond to the ultrasonic waves reflected by the target object. The control system may be configured to detect an artery within the target object and to estimate a BP within the artery based, at least in part, on ultrasonic receiver signals from the single array of M ultrasonic receiver elements.

Particular implementations of the subject matter described in this disclosure can be implemented to realize one or more of the following potential advantages. Some disclosed ultrasonic-based devices for BP estimation are capable of estimating—e.g., monitoring—BP according to single-sensor measurement. In some implementations, a single ultrasonic sensor array may be configured to estimate both PWV and the area of an artery, thus enabling the monitoring of BP with measurements at a single arterial location. Accordingly, such devices include relatively less hardware than previously-deployed ultrasonic-based devices for BP estimation and have relatively less complexity and lower costs. Some disclosed ultrasonic-based devices for BP estimation do not rely on the methods for PWV estimation that were used by previously-disclosed devices and may provide more accurate measurements and BP estimates. The disclosed ultrasonic-based devices for BP estimation are suitable for compact form-factors, which is especially desirable for wearable devices such as smart watches or other wearable health-monitoring or fitness-monitoring devices.

According to some implementations, the control system may be configured to apply a receiver-side beamforming process to ultrasonic receiver signals received from each receiver element of the single array of ultrasonic receiver elements. In some implementations, the control system may be configured to estimate a change in cross-sectional area of the artery based, at least in part, on the beamformed ultrasonic receiver image. Such configurations include ultrasonic-capable devices that can provide a higher SNR—as compared to previously-deployed devices having a single receiver element—for signals corresponding to the ultrasonic response of one or more arterial walls. According to some implementations, the control system may be configured to estimate BP based, at least in part, on the change in the cross-sectional area of the artery. Signals obtained from multiple receiver elements of an ultrasonic receiver array can provide relatively more accurate information about the area of an artery that would not be available if, for example, signals were obtained from only a single ultrasonic receiver element.

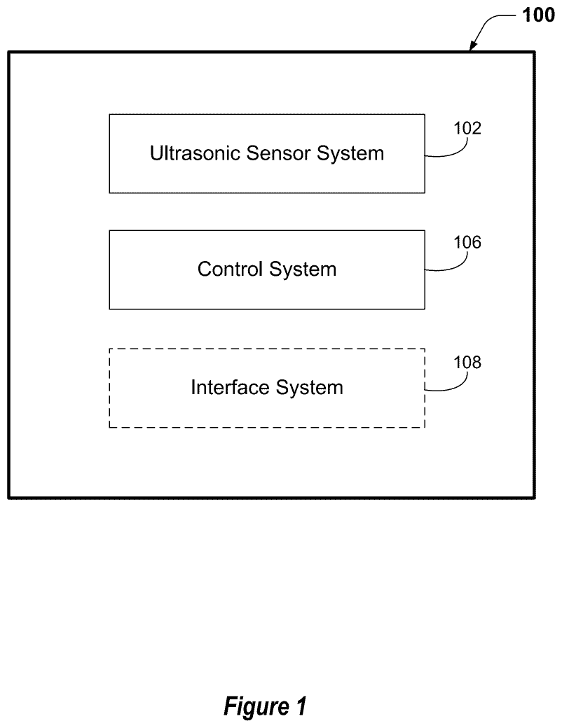

FIG. 1 is a block diagram that shows example components of an apparatus according to some disclosed implementations. In this example, the apparatus 100 includes an ultrasonic sensor system 102 and a control system 106. According to some examples, the apparatus 100 may be configured to allow measurements for BP estimation to be obtained from a single arterial location. Some implementations of the apparatus 100 may include an interface system 108. As with other disclosed implementations, in some alternative implementations the apparatus 100 may include more components, fewer components or different components.

In this example, the ultrasonic sensor system 102 is configured to transmit ultrasonic waves and to receive ultrasonic waves reflected by a target object. According to some examples, the ultrasonic sensor system 102 includes an array of ultrasonic receiver elements. In some examples, the ultrasonic sensor system 102 includes a single array of M ultrasonic receiver elements. According to some examples, the single array of M ultrasonic receiver elements may be linearly arranged, or in other words arranged along a line. In some examples, the ultrasonic sensor system 102 may be, or may include, an array of electrodes arranged on a piezoelectric layer, such as a layer of PVDF polymer, a layer of PVDF-TrFE copolymer, or a layer of piezoelectric composite material. According to some such examples, the same piezoelectric layer may be configured to transmit ultrasonic waves and to receive ultrasonic waves reflected by a target object. In some implementations, other piezoelectric materials may be used in the piezoelectric layer, such as aluminum nitride (AlN) or lead zirconate titanate (PZT). The ultrasonic sensor system 102 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. In some examples, the apparatus 100 may include one or more separate ultrasonic transmitter elements. In some such examples, the ultrasonic transmitter(s) may include an ultrasonic plane-wave generator.

The control system 106 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 106 also may include (and/or be configured for communication with) one or more memory devices, such as one or more random access memory (RAM) devices, read-only memory (ROM) devices, etc. Accordingly, the apparatus 100 may have a memory system that includes one or more memory devices, though the memory system is not shown in FIG. 1. The control system 106 may be configured for controlling the ultrasonic sensor system 102. The control system 106 may be configured for receiving and processing data from the ultrasonic sensor system 102, e.g., as described below. In some implementations, functionality of the control system 106 may be partitioned between one or more controllers or processors, such as a dedicated sensor controller and an applications processor of a mobile device.

In some examples, the control system 106 may be configured to control the ultrasonic sensor system to transmit ultrasonic waves to a target object on an outer surface of the apparatus. In some such examples, the control system 106 may be configured to receive ultrasonic receiver signals from each of a plurality of ultrasonic receiver elements in a single array of M ultrasonic receiver elements of the ultrasonic sensor system 102, where M is an integer of two or more. The ultrasonic receiver signals may correspond to the ultrasonic waves reflected from the target object.

According to some examples, the control system 106 may be configured to detect an artery within the target object based on the ultrasonic receiver signals. In some examples, the control system 106 may be configured to estimate a blood pressure within the artery based on ultrasonic receiver signals from the single array of M ultrasonic receiver elements.

In some examples, the control system 106 may be configured to apply a receiver-side beamforming process to the ultrasonic receiver signals, to produce a beamformed ultrasonic receiver image. According to some examples, the control system 106 may be configured to estimate a change in cross-sectional area of the artery based, at least in part, on the beamformed ultrasonic receiver image. In some such examples, the control system 106 may be configured to estimate blood pressure based, at least in part, on the change in the cross-sectional area of the artery.

Some implementations of the apparatus 100 may include the interface system 108. In some examples, the interface system 108 may include a wireless interface system. In some implementations, the interface system 108 may include a user interface system, one or more network interfaces, one or more interfaces between the control system 106 and a memory system and/or one or more interfaces between the control system 106 and one or more external device interfaces (e.g., ports or applications processors), or combinations thereof. According to some examples in which the interface system 108 is present and includes a user interface system, the user interface system may include a microphone system, a loudspeaker system, a haptic feedback system, a voice command system, one or more displays, or combinations thereof. According to some examples, the interface system 108 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

The apparatus 100 may be used in a variety of different contexts, some examples of which are disclosed herein. For example, in some implementations a mobile device, such as a hand-held device, may include the apparatus 100. In some implementations, a wearable device may include the apparatus 100. The wearable device may, for example, be a bracelet, an armband, a wristband, a watch, a ring, a headband or a patch. Accordingly, in some examples the apparatus 100 may be configured to be worn by, or attached to, a person.

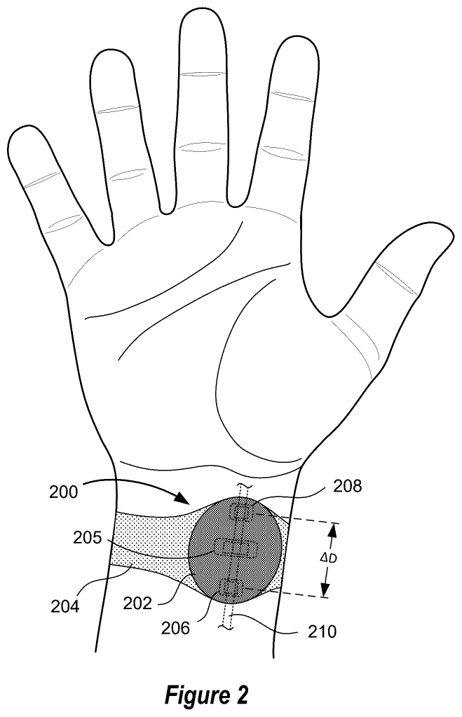

FIG. 2 shows an example of a previously-deployed ultrasonic-based device for BP estimation. In this example, the monitoring device 200 is designed to be worn around a human wrist. In the illustrated example, the monitoring device 200 includes a housing 202 that is integrally formed with, coupled to or otherwise integrated with a wristband 204. The sensors 206 and 208 may, in some instances, each include portions of an ultrasonic receiver system. The sensor 205 may, in some instances, include a portion of an ultrasonic receiver system and an instance of a light source system. In this example, the monitoring device 200 is coupled around the wrist such that the sensors 205, 206 and 208 within the housing 202 are each positioned along a segment of the radial artery 210 (note that the sensors are generally hidden from view from the external or outer surface of the housing facing the subject—in this example, a portion of a person's wrist—while the monitoring device is coupled with the subject, but exposed on an inner surface of the housing to enable the sensors to obtain measurements from the underlying artery through the subject's skin).

According to this example, the sensors 206 and 208 are configured to measure PWV of blood within the radial artery 210, whereas the sensor 205 is configured to measure changes in the cross-sectional area of the radial artery 210. Based at least in part on these measurements, the monitoring device 200 is designed to estimate blood pressure.

As shown in FIG. 2, the sensors 206 and 208 are separated by a fixed distance ΔD. As noted elsewhere herein, with a relatively small ΔD between two ultrasonic receiver portions that is feasible for a single device with a smart watch form factor has resulted error-prone PWV estimations. Providing a single device that had ultrasonic receivers spaced far enough apart for reasonably accurate PWV measurements required a large form factor, which was not acceptable for some consumers. Moreover, BP estimation should be based on accurate measurements from the same artery. Having a sensor element—such as the sensor 205—and 2 ultrasonic receivers with a sufficiently large ΔD for accurate PWV determination in a single wearable device causes potential challenges with aligning all three sensor elements along a single artery.

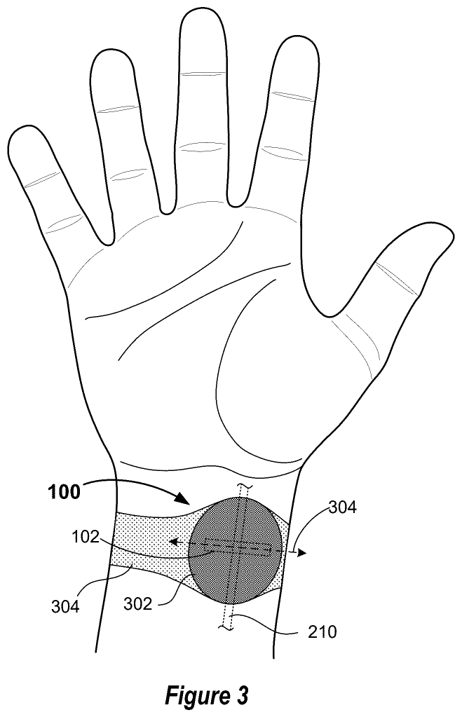

FIG. 3 shows an example of a ultrasonic-based device for BP estimation according to the present disclosure. The apparatus 100 of FIG. 3 is an instance of the apparatus 100 of FIG. 1. Although not shown in FIG. 3, the apparatus 100 includes an instance of the control system 106. In some instances, the apparatus 100 may include an interface system 108. As with other disclosed examples, the type, number, size and arrangement of elements shown in FIG. 3 and described herein are merely examples. Other implementations may include different types of elements, numbers of elements, arrangements of elements, or combinations thereof.

In this example, the apparatus 100 is designed to be worn around a human wrist and includes a housing 302 that is integrally formed with, coupled to or otherwise integrated with a wristband 304. According to this example, the outward appearance of the apparatus 100 is like that of the monitoring device 200 of FIG. 2. However, instead of having separate sensors 205, 206 and 208, as shown in the example of FIG. 2, the apparatus 100 includes a single instance of the ultrasonic sensor system 102. In this example, apparatus 100 is configured to estimate both PWV and the area of the radial artery 210—including changes in the area of the radial artery 210—thus enabling the monitoring of BP with measurements at a single arterial location. Taking measurements at a single arterial location can improve the accuracy of such measurements and therefore can improve the accuracy of corresponding BP estimations.

According to this example, the ultrasonic sensor system 102 includes a single array of M ultrasonic receiver elements, M being an integer of 2 or more. In some instances, M may be 2, 3, 4, 5, 6, 7, 8, 9, 10, 11, 12, etc. In this example, the single array of M ultrasonic receiver elements extends along an axis 304 that is configured to extend across the radial artery 210. One could consider the “single arterial location” at which measurements are taken to be the intersection of the axis 304 and the radial artery 210.

The novel apparatus 100 shown in FIG. 3 is made possible, at least in part, by different measurement and BP calculation techniques that are disclosed herein. Unlike previously-deployed ultrasonic-based devices for BP estimation, the apparatus 100 does not require two separate ultrasonic receiver portions for PWV estimations. Accordingly, the apparatus 100 includes relatively less hardware than previously-deployed ultrasonic-based devices for BP estimation, has relatively less complexity and may potentially be manufactured at a lower cost. As described in more detail below, the apparatus 100 does not rely on the methods for PWV estimation that were used by previously-disclosed devices and may provide more accurate measurements and BP estimates. The apparatus 100 is suitable for compact form-factors, which is highly desirable for wearable devices such as smart watches or other wearable health-monitoring or fitness-monitoring devices. In some other implementations, the apparatus 100 may be designed or adapted for positioning around a forearm, an upper arm, an ankle, a lower leg, an upper leg, an car or a finger using one or more straps, bands, etc. In some alternative implementations, the apparatus 100 may be implemented in a wearable patch, which in some examples may be adhesively attached to a portion of the human body.

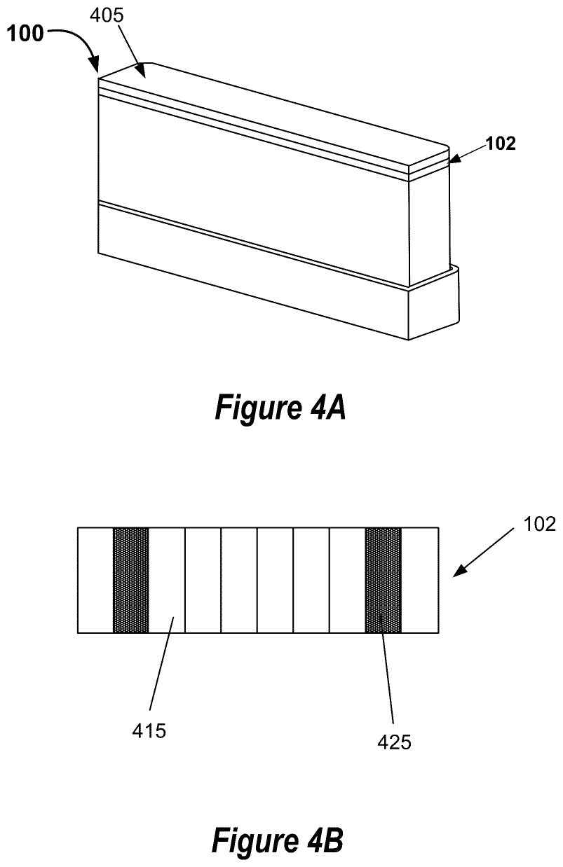

FIG. 4A show examples of an apparatus that includes an ultrasonic sensor system. FIG. 4B shows a top view of the ultrasonic sensor system 102 of FIG. 4A. The apparatus 100 of FIG. 4A is an instance of the apparatus 100 of FIG. 1. In the example of FIG. 4A, only an edge of the ultrasonic sensor system 102 is visible. The ultrasonic sensor system 102 of FIG. 4B is an example of the ultrasonic sensor system 102 shown in FIG. 3. As with other disclosed examples, the type, number, size and arrangement of elements shown in FIG. 4 and described herein are merely examples. Other implementations may include different types of elements than are shown in FIG. 4A or 4B, different numbers of elements than are shown in FIG. 4A or 4B, different arrangements of elements than are shown in FIG. 4A or 4B, or combinations thereof. For example, in some instances the apparatus 100 may include an interface system 108, although this is not shown in FIG. 4A or 4B.

According to this example, the ultrasonic sensor system 102 includes a single array of M active ultrasonic receiver elements 415 and two inactive ultrasonic receiver elements 425. In this example, M=6. In some alternative examples, M may be 2, 3, 4, 5, 7, 8, 9, 10, 11, 12, etc. In some examples, the apparatus 100 of FIG. 4A may be configured such that the single array of M active ultrasonic receiver elements extends along an axis that is configured to extend across an artery, such as the ultrasonic sensor system 102 in FIG. 3 that is shown extending along the axis 304 across the radial artery 210.

Although not shown in FIG. 4A or FIG. 4B, the apparatus 100 includes an instance of the control system 106 that is configured to control the ultrasonic sensor system 102 to transmit ultrasonic waves and to receive ultrasonic receiver signals from each of the ultrasonic receiver elements 415 of the ultrasonic sensor system 102. In some instances, the ultrasonic receiver signals may correspond to ultrasonic waves reflected from one or more portions of a target object, such as arterial walls, the blood within an artery, or combinations thereof.

In this example, the ultrasonic sensor system 102 is configured to transmit ultrasonic waves through the outer surface 405 of the apparatus 100 and to receive reflected ultrasonic waves from a target object through the outer surface 405. The apparatus 100 may, in some implementations, be configured such that the outer surface 405 will reside proximate a portion of a human body—such as a wrist, a finger, a leg, etc. —when the apparatus 100 is worn.

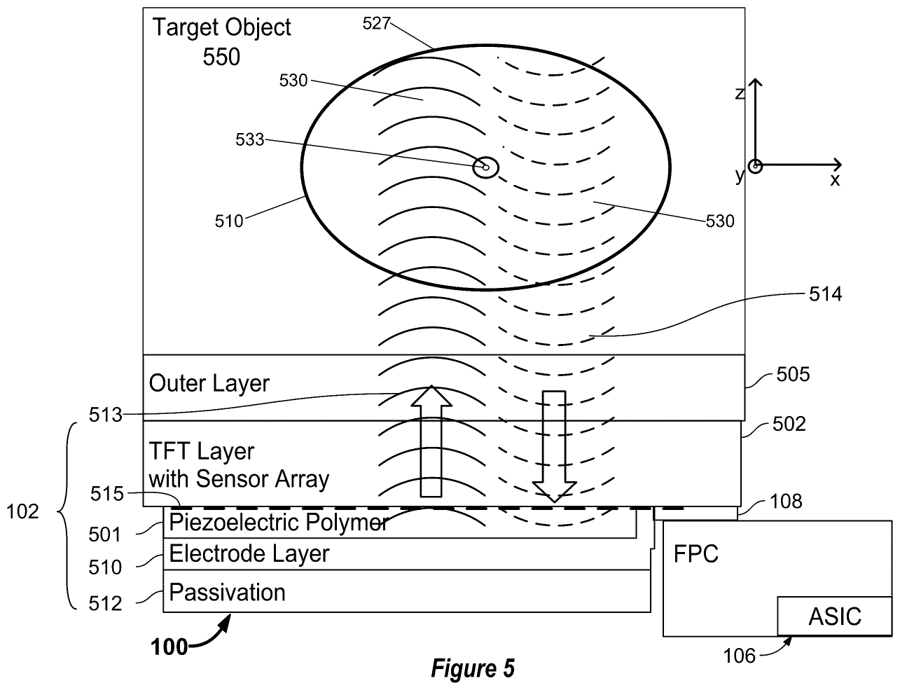

FIG. 5 shows example components of an apparatus according to some disclosed implementations. As with other disclosed implementations, the types, number and arrangement of elements, as well as the dimensions of elements, are merely examples. In this example, the apparatus 100 of FIG. 5 is an instance of the apparatus 100 of FIG. 1, an instance of the apparatus 100 of FIG. 3 and an instance of the apparatus 100 of FIG. 4A. According to this example, the ultrasonic sensor system 102 of FIG. 5 is an instance of the ultrasonic sensor system 102 of FIG. 1, an instance of the ultrasonic sensor system 102 of FIG. 3 and an instance of the ultrasonic sensor system 102 of FIG. 4B. In this example, the apparatus 100 is configured to perform at least some of the methods disclosed herein.

According to this implementation, the ultrasonic sensor system 102 includes an ultrasonic transceiver layer 501, an electrode layer 510 on one side of the ultrasonic transceiver layer 501 and an array of receiver elements 515 on a second and opposing side of the ultrasonic transceiver layer 501. In this implementation, the ultrasonic transceiver layer 501 includes one or more piezoelectric polymers. In other implementations, the ultrasonic transceiver layer 501 may include other types of piezoelectric materials.

According to this example, the electrode layer 510 resides between a passivation layer 512 and the ultrasonic transceiver layer 501. In some examples, the passivation layer 512 may include an adhesive, such as an epoxy film, a polymer layer (such as a polyethylene terephthalate (PET) layer), etc.

In this example the thin-film transistor (TFT) layer 502 includes a TFT substrate and circuitry for the array of M receiver elements 515. In this example, M=16 and the array of M receiver elements 515 is a linear array. The TFT layer 502 may be a type of metal-oxide-semiconductor field-effect transistor (MOSFET) made by depositing thin films of an active semiconductor layer as well as a dielectric layer and metallic contacts over a TFT substrate. In some examples, the TFT substrate may be a non-conductive material such as glass.

According to this implementation, the TFT layer 502, the array of receiver elements 515 and the electrode are electrically coupled to at least a portion of the control system 106 and one side of the ultrasonic transceiver layer 501 via a portion of the interface system 108, which includes electrically conducting material and a flexible printed circuit (FPC) in this instance.

In this example, the apparatus 100 is configured to perform at least some of the methods disclosed herein. In this example, the control system 106—which is an instance of the control system 106 of FIG. 1—is configured to control the ultrasonic sensor system—more specifically, the ultrasonic transceiver layer 501—to transmit one or more ultrasonic waves 513. Although the ultrasonic waves 513 are shown having arcuate wave fronts, in some examples the ultrasonic waves 513 may be plane waves, or substantially planar waves, e.g. parallel to or substantially parallel to (e.g., within 5 degrees of being parallel, within 10 degrees of being parallel, within 15 degrees of being parallel, etc.) the ultrasonic transceiver layer 501. According to this example, the ultrasonic waves 513 are transmitted through the TFT layer 502 and the outer layer 505 into a target object 550, which includes an artery portion 510 having an axis 533 that is substantially parallel to (e.g., within 5 degrees of being parallel, within 10 degrees of being parallel, within 15 degrees of being parallel, etc.) the y axis and therefore is substantially perpendicular to the axis along which the linear array of ultrasonic receiver elements 515 is arranged. According to this example, some of the ultrasonic waves 513 have reached the arterial wall 527 and has caused the arterial wall 527 to reflect the ultrasonic waves 514, at least some of which are received by the linear array of ultrasonic receiver elements 515. Additional ultrasonic waves may be reflected from the arterial wall closer to the apparatus 100, from blood within the artery 510, or combinations thereof. Corresponding ultrasonic receiver signals may be provided to the control system 106.

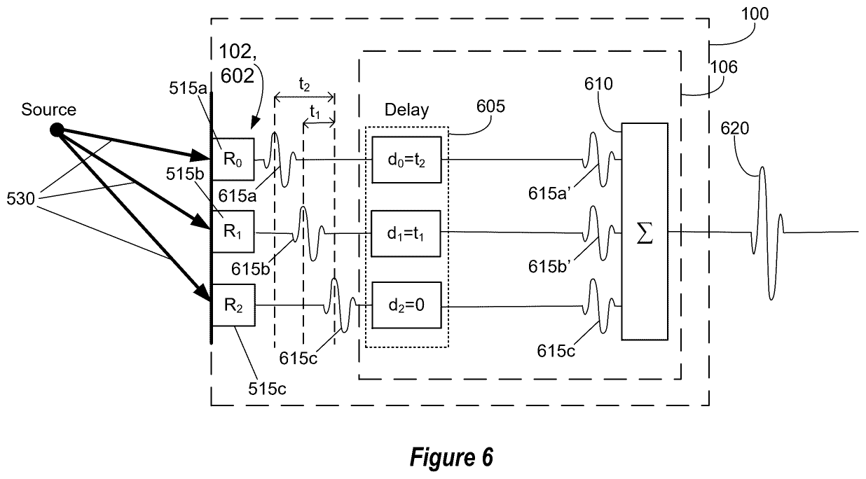

FIG. 6 shows an example of an apparatus that is configured to perform a receiver-side beamforming process. In this example, the receiver-side beamforming process is a delay-and-sum beamforming process. As with other disclosed examples, the types, numbers, sizes and arrangements of elements shown in FIG. 6 and described herein, as well as the associated described methods, are merely examples.

In this example, a source is shown emitting ultrasonic waves 530, which are detected by active ultrasonic receiver elements 515a, 515b and 515c of an array of ultrasonic receiver elements 602. The array of ultrasonic receiver elements 602 is part of an ultrasonic sensor system 102. The ultrasonic waves 530 may, in some examples, correspond to ultrasonic waves reflected from a target object. In this example, the active ultrasonic receiver elements 515a, 515b and 515c provide ultrasonic receiver signals 615a, 615b and 615c, respectively, to the control system 106.

According to this example, the control system 106 includes a delay module 605 and a summation module 610. In this example, the delay module 605 is configured to determine whether a delay should be applied to each of the ultrasonic receiver signals 615a, 615b and 615c, and if so, what delay to apply. According to this example, the delay module 605 determines that a delay d₀ of t₂ should be applied to the ultrasonic receiver signal 615a, that a delay d₁ of t₁ should be applied to the ultrasonic receiver signal 615b and that no delay should be applied to the ultrasonic receiver signal 615c. Accordingly, the delay module 605 applies a delay of t₂ to the ultrasonic receiver signal 615a, producing the ultrasonic receiver signal 615a′, and applies a delay of t₁ to the ultrasonic receiver signal 615b, producing the ultrasonic receiver signal 615b′.

In some examples, the delay module 605 may determine what delay, if any, to apply to an ultrasonic receiver signal by performing a correlation operation on input ultrasonic receiver signals. For example, the delay module 605 may perform a correlation operation on the ultrasonic receiver signals 615a and 615c, and may determine that by applying a time shift of t₂ to the ultrasonic receiver signal 615a, the ultrasonic receiver signal 615a would be strongly correlated with the ultrasonic receiver signal 615c. Similarly, the delay module 605 may perform a correlation operation on the ultrasonic receiver signals 615b and 615c, and may determine that by applying a time shift of t₁ to the ultrasonic receiver signal 615b, the ultrasonic receiver signal 615b would be strongly correlated with the ultrasonic receiver signal 615c.

According to this example, the summation module 610 is configured to sum the ultrasonic receiver signals 615a′, 615b′ and 615c, producing the summed signal 620. One may observe that the amplitude of the summed signal 620 is greater than the amplitude of any one of the ultrasonic receiver signals 615a, 615b or 615c. In some instances, the signal-to-noise ratio (SNR) of the summed signal 620 may be greater than the SNR of any of the ultrasonic receiver signals 615a, 615b or 615c.

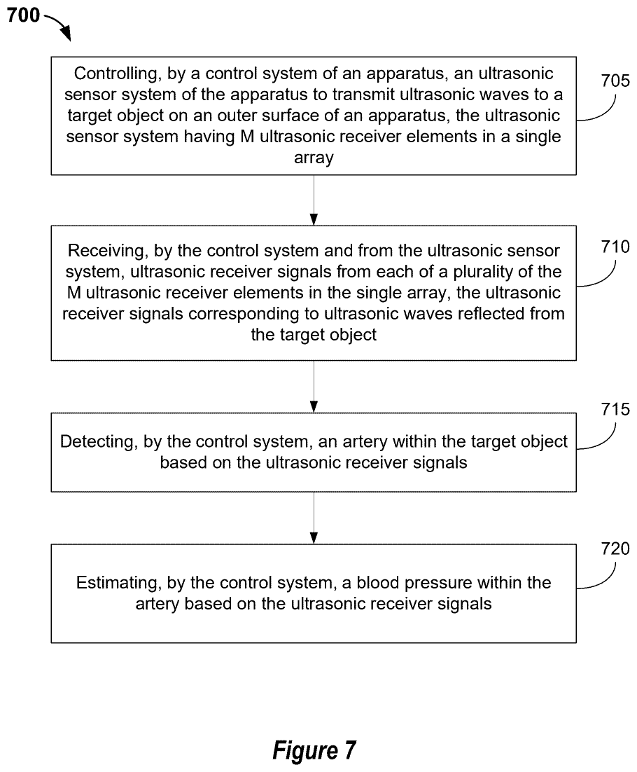

FIG. 7 is a flow diagram that shows examples of some disclosed operations. The blocks of FIG. 7 may, for example, be performed by the apparatus 100 of any one of FIG. 1, 3, 4A or 5, or by a similar apparatus. As with other methods disclosed herein, the method outlined in FIG. 7 may include more or fewer blocks than indicated. Moreover, the blocks of methods disclosed herein are not necessarily performed in the order indicated. In some instances, one or more of the blocks shown in FIG. 7 may be performed concurrently.

In this example, block 705 involves controlling, by a control system of an apparatus, an ultrasonic sensor system of the apparatus to transmit ultrasonic waves to a target object on an outer surface of the apparatus. According to this example, the ultrasonic sensor system has M ultrasonic receiver elements in a single array. In some examples, M may be an integer of 2 or more. In some instances, M may be 2, 3, 4, 5, 6, 7, 8, 9, 10, 11, 12, etc. In some such examples, the single array of M ultrasonic receiver elements may be linearly arranged. The ultrasonic receiver elements may be elements of the ultrasonic sensor system 102, such as the ultrasonic receiver elements 415 of FIG. 4 or FIG. 5. The target object may be a portion of a finger, a wrist, an arm, a leg, etc., depending on the particular example. The control system may be an instance of the control system 106.

According to this example, block 710 involves receiving, by the control system, ultrasonic receiver signals from each of M ultrasonic receiver elements in a single array. In this example, the ultrasonic receiver signals correspond to ultrasonic waves reflected from the target object. In some examples, the ultrasonic receiver signals may correspond to ultrasonic waves generated by an arterial wall—such as the arterial wall 527 that is shown in FIG. 5—to blood within an artery, or both.

According to this example, block 715 involves detecting, by the control system, an artery within the target object based on the ultrasonic receiver signals. In some examples, the artery may be a radial artery, such as the radial artery 210 of FIG. 3. According to some such examples, the apparatus may be configured to be worn on a human wrist, as in the example of the apparatus 100 of FIG. 3. In other examples, the artery may be another type of artery, such as an artery inside an upper arm, a leg, a neck, an car, etc. In some examples, detecting the artery may involve applying image recognition software, for example software that is trained to detect ultrasonic receiver signals corresponding to arterial walls, ultrasonic receiver signals corresponding to blood within an artery, etc. In some examples, detecting the artery may involve applying a range gate delay (RGD) corresponding to a likely arterial depth within the target object, a range gate window (RGW) corresponding to a likely range of arterial depths within the target object, etc.

In this example, block 720 involves estimating, by the control system, a blood pressure within the artery based on the ultrasonic receiver signals. According to some examples, method 700 may involve estimating a change in cross-sectional area of the artery based at least in part the ultrasonic receiver signals from the single array of M ultrasonic receiver elements. In some such examples, estimating the blood pressure may be based, at least in part, on the change in the cross-sectional area of the artery. In some examples, method 700 may involve applying, by the control system, a receiver-side beamforming process to the ultrasonic receiver signals from the single array of M ultrasonic receiver elements, to produce a beamformed ultrasonic receiver image. In some such examples, estimating a cross-sectional area of the artery, a change in the cross-sectional area of the artery, or both, may be based at least in part on the beamformed ultrasonic receiver image.

According to some examples, method 700 may involve estimating, by the control system, a change in a blood flow rate within the artery based on the ultrasonic receiver signals from the single array of M ultrasonic receiver elements. In some such examples, estimating the blood pressure may be based, at least in part, on the change in the blood flow rate within the artery. Accordingly, in some examples, estimating the blood pressure may be based at least in part on the change in the blood flow rate within the artery and the change in cross-sectional area of the artery.

According to some examples, the ultrasonic receiver signals may include frequencies in a range from 10 MHz to 25 MHz. According to some examples, the control system may be configured to receive ultrasonic receiver signals from the ultrasonic sensor system at a frame rate in a range from 1 KHz to 3 KHz. Such frequencies and frame rates can provide sufficiently accurate measurements of the blood flow rate within the artery, and the change in the blood flow rate within the artery, for reliable BP estimation.

In some examples, method 700 may involve estimating, by the control system, the change in the blood flow rate within the artery based at least in part on speckle decorrelation (SDC)-based blood flow measurements. SDC can be used to measure blood flow velocity profiles. As scatterers travel through an ultrasound imaging plane in the elevational direction (the blood flow direction), the received echo signals decorrelate at a rate related to the flow velocity. In the example shown in FIG. 5, the “elevational direction” is along the y axis and the ultrasonic receiver elements 515 are arranged along the x axis.

The correlation function of echo signal intensities in the elevational direction for two scatterers can be expressed as follows:

$${R}_{I} ( Δ r ) = ( {I}_{1} {I}_{2} ) = {〈 I 〉}^{2} ( 1 + {‖ ρ ( Δ r ) ‖}^{2} ) .$$

In the foregoing equation, R_{I}(Δr) represents the correlation between echo signal intensities I₁ and I₂ in the elevational direction, Δr represents the spatial distance between I₁ and I₂ that can be varied to lag in time when the scatterer is moving across time, <.> represents the expectation of a random variable, <I> represents the mean intensity that is constant with respect to position and ∥ρ(Δr)∥² represents the normalized coherence factor related to the system point spread function. In high frame rate ultrasonic beamformed images, singular value decomposition (SVD) can be applied to the ultrasonic signals to filter out unwanted artifacts. The movement of particles will cause the SVD filtered ultrasonic signals to fluctuate at a rate that is proportional to the flow speed. Speckle patterns in B-mode images produced by acoustic wave interference decorrelate as the relative positions between the scatterers and the ultrasonic sensor location change.

Some relevant examples of speckle decorrelation-based blood flow measurements are disclosed in D. Park et al., “Ultrasound Speckle Decorrelation-Based Blood Flow Measurements,” (Ultrasound in Medicine & Biology, Volume 49, June 2023, Pages 1491-1498), which is hereby incorporated by reference and for all purposes.

Alternatively, or additionally, method 700 may involve estimating, by the control system, the change in the blood flow rate within the artery based at least in part on a Doppler-based method. Doppler-based methods generally require that at least some ultrasonic waves are directed along the artery. For example, referring to FIG. 5, a Doppler-based method would involve directing at least some ultrasonic waves out of the x-z plane, such that ultrasonic waves are at least partially directed along the positive or negative y axis. The primary components of blood responsible for generating the Doppler effect are red blood cells, the size of which (6-8 um) is much smaller than the wavelength of ultrasound (e.g., in the range of 100-500 um), causing the red blood cells to act as Rayleigh scatterers of transmitted ultrasonic waves. Some Doppler-based methods may involve a transmit-side beamforming process that involves causing constructive interference of ultrasonic waves at a desired angle, or angle range, in the y-z plane. In some such examples, the transmit-side beamforming process may involve causing constructive interference of ultrasonic waves in a Doppler angle range of 30 degrees to 60 degrees, where the Doppler angle Θ is measured between the direction of the ultrasonic waves and the blood flow direction, the latter of which corresponds with the y axis shown in FIG. 5. The measured Doppler shift f_{D}—which is the difference between the frequency of the transmitted ultrasound f_{T} and the frequency of the received ultrasound f_{R}—is directly proportional to the cosine of the Doppler angle according to the following equation:

$${f}_{D} = {f}_{R -} {f}_{T} = \frac{2 {f}_{T} V cos Θ}{c}$$

In the foregoing equation, V represents the blood flow velocity and c represents the speed of sound. Accordingly, the blood flow velocity V may be determined as follows:

$$V = \frac{c {f}_{D}}{2 {f}_{T} cos Θ}$$

According to some such examples, blood flow velocity may be measured according to one or more methods disclosed in F. Wang et al., “Flexible Doppler ultrasound device for the monitoring of blood flow velocity,” (Science Advances 7: eabi9283, 27 Oct. 2021), which is hereby incorporated by reference and for all purposes.

In some implementations, the apparatus performing the method 700 may include a magnetic sensor system. According to some such examples, method 700 may involve estimating a change in a blood flow rate within the artery based on magnetic sensor signals from the magnetic sensor system. For example, the magnetic blood flow sensor may include a permanent magnet placed in the vicinity of a blood vessel. As the blood flows through the vessel, the magnetic field is modified due to hemoglobin in the blood and is sensed by measuring the induced electromotive force (EMF) or magnetic field attributes. If one assumes that blood flow velocity is symmetrical in relation to the blood vessel's longitudinal axis, the average blood flow velocity Vₘ may be expressed as follows:

$${V}_{m} = \frac{E}{B 2 r}$$

In the foregoing equation, E represents the induced electromotive force (EMF), which is proportional to an induced magnetic field, B represents the normal component of the magnetic field and r represents the radius of the blood vessel.

In some implementations, the apparatus performing the method 700 may include an optical sensor system. In some such examples, method 700 may involve estimating a change in a blood flow rate within the artery based on optical sensor signals from the optical sensor system. According to some examples, blood flow velocity may be measured according to one or more methods disclosed in A. Zhang et al., “Non-Invasive Blood Flow Speed Measurement Using Optics,” (Sensors 2022, 22, 897 (MDPI)), which is hereby incorporated by reference and for all purposes. In some such examples, the blood flow velocity V may be determined according to the following equation:

$$\frac{1}{{T}_{F}} \approx {Vnk}_{0} \sqrt{\frac{{μ}_{s}^{′ in}}{3 {μ}_{β}^{in}}}$$

In the foregoing equation, which is Equation 17 of the Zhang paper, n represents the refractive index of blood, ko represents the wavevector of 784 nm light in vacuum, μₛ′ⁱⁿ represents the reduced scattering coefficient of blood (m⁻¹), and μₐⁱⁿ represents the absorption coefficient of blood (m⁻¹). If one defines T_{F} as the characteristic decorrelation time, and 1/T_{F} as the characteristic decorrelation rate, one can determine the key relationship that the characteristic decorrelation rate is only a function of flow speed and blood optical parameters, as shown in the foregoing equation. The wavelength of the light source is preferably chosen to penetrate the tissue and to provide a good contrast for blood absorption compared to the surrounding tissue. For example, an 808 nm to 1050 nm infrared light (IR) source can be used.

As noted above, estimating the blood pressure may, in some examples, be based at least in part on the change in the blood flow rate within the artery and the change in cross-sectional area of the artery. In some such examples, estimating the blood pressure may be based in part on a derivative of the blood flow rate (Q) within the artery with respect to the cross-sectional area (A) of the artery. This derivative may be expressed as dQ/dA.

Following is a brief explanation of some underlying theories that have been developed by the present inventors in which BP estimation is based, in part, on dQ/dA.

According to the Bramwell-Hill equation, pulse wave velocity (PWV) is directly related to compliance, as follows:

$$PWV = \sqrt{\frac{A}{ρ} \frac{d P}{d A}} . \qquad ( 1 )$$

In Equation (1), A represents the cross-sectional area of an artery, P represents the blood pressure in the artery, and p represents the density of blood in the artery. We can rewrite Equation (1) to generate a pressure waveform—assuming PWV is relatively constant during a cardiac cycle—as follows:

$$P ( t ) - {P}_{0} = ρ {PWV}^{2} ln ( \frac{A ( t )}{{A}_{0}} ) , \qquad ( 2 )$$

In Equation (2), P₀ represents the blood pressure at arterial area A₀.

Some disclosed “flow/area” methods involve directly estimating a local PWV based on the change of blood flow rate with respect to the change in arterial area during a cardiac cycle. The characteristic impedance of a blood vessel, such as an artery, may be expressed as follows:

$${Z}_{c} = \frac{d P}{d Q} = \sqrt{\frac{ρ}{A} \frac{d P}{d A}} . \qquad ( 3 )$$

In Equation (3), Q represents the blood flow rate within a blood vessel.

By combining Equations (1) and (3), PWV may be expressed as follows:

$${PWV}_{QA} = \sqrt{\frac{A}{ρ} \frac{d P}{d A}} = \frac{1}{{Z}_{c}} \frac{d P}{d A} = \frac{d Q}{d P} \frac{d P}{d A} = \frac{d Q}{d A} . \qquad ( 4 )$$

We have disclosed various methods for calculating the how the area of a blood vessel varies during the cardiac cycle. Such methods can be used to obtain dA. For example, as noted elsewhere herein, in some examples method 700 may involve applying, by the control system, a receiver-side beamforming process to the ultrasonic receiver signals from the single array of M ultrasonic receiver elements, to produce a beamformed ultrasonic receiver image. In some such examples, estimating a cross-sectional area of the artery, a change in the cross-sectional area of the artery, or both, may be based at least in part on the beamformed ultrasonic receiver image. Accordingly, in some such examples, dA may be based, at least in part, on beamformed ultrasonic receiver images of an arterial cross-sectional area.

Similarly, we have disclosed various methods for calculating how the rate of blood flow in a blood vessel varies during the cardiac cycle based on measurements at a single arterial location using an ultrasonic sensor system. These methods include, but are not limited to, including speckle decorrelation (SDC)-based blood flow measurements and Doppler-based methods. Such methods can be used to obtain dQ.

After dA and dQ are obtained, PWV may be estimated using Equation 4 according to some examples. According to some such examples, after PWV has been estimated, BP may be estimated using Equation (2) along with measurements of A(t), which represents how the arterial area varies over time.

Implementation examples are described in the following numbered clauses:

1. An apparatus, including: an ultrasonic sensor system including a single array of M ultrasonic receiver elements; and a control system configured to: control the ultrasonic sensor system to transmit ultrasonic waves to a target object on an outer surface of the apparatus; receive, from the ultrasonic sensor system, ultrasonic receiver signals from each of a plurality of the M ultrasonic receiver elements in the single array, the ultrasonic receiver signals corresponding to ultrasonic waves reflected from the target object; detect an artery within the target object based on the ultrasonic receiver signals; and estimate a blood pressure within the artery based on the ultrasonic receiver signals.

2. The apparatus of clause 1, where the apparatus is configured to be worn on a human wrist and where the artery is a radial artery.

3. The apparatus of clause 1 or clause 2, where the control system is further configured to apply a receiver-side beamforming process to the ultrasonic receiver signals, to produce a beamformed ultrasonic receiver image.

4. The apparatus of clause 3, where: the control system is further configured to estimate a change in cross-sectional area of the artery based at least in part on the beamformed ultrasonic receiver image; and estimating the blood pressure is based in part on the change in the cross-sectional area of the artery.

5. The apparatus of clause 4, where: the control system is further configured to estimate a change in a blood flow rate within the artery based on the ultrasonic receiver signals; and estimating the blood pressure is based in part on the change in the blood flow rate within the artery and the change in cross-sectional area of the artery.

6. The apparatus of clause 5, where estimating the blood pressure is based in part on a derivative of the blood flow rate (Q) within the artery with respect to the cross-sectional area (A) of the artery (dQ/dA).

7. The apparatus of clause 6, where the control system is configured to estimate a pulse wave velocity based on the derivative dQ/dA.

8. The apparatus of any one of clauses 5-7, where the control system is configured to estimate the change in the blood flow rate within the artery based at least in part on speckle decorrelation-based blood flow measurements or a Doppler-based method.

9. The apparatus of any one of clauses 5-7, further including a magnetic sensor system, where: the control system is further configured to estimate a change in a blood flow rate within the artery based on magnetic sensor signals from the magnetic sensor system; and estimating the blood pressure is based in part on the change in the blood flow rate within the artery and the change in cross-sectional area of the artery.

10. The apparatus of any one of clauses 5-7, further including an optical sensor system, where: the control system is further configured to estimate a change in a blood flow rate within the artery based on optical sensor signals from the optical sensor system; and estimating the blood pressure is based in part on the change in the blood flow rate within the artery and the change in cross-sectional area of the artery.

11. The apparatus of any one of clauses 1-10, where the ultrasonic sensor system is configured to provide ultrasonic receiver signals to the control system at a frame rate in a range from 1 KHz to 3 KHz.

12. The apparatus of any one of clauses 1-11, where the ultrasonic receiver signals include frequencies in a range from 10 MHz to 25 MHz.

13. The apparatus of any one of clauses 1-12, where the single array of M ultrasonic receiver elements is linearly arranged.

14. An apparatus, including: an ultrasonic sensor system including a single array of M ultrasonic receiver elements; and control system means for: controlling the ultrasonic sensor system to transmit ultrasonic waves to a target object on an outer surface of the apparatus; receiving, from the ultrasonic sensor system, ultrasonic receiver signals from each of a plurality of the M ultrasonic receiver elements in the single array, the ultrasonic receiver signals corresponding to ultrasonic waves reflected from the target object; detecting an artery within the target object based on the ultrasonic receiver signals; and estimating a blood pressure within the artery based on the ultrasonic receiver signals.

15. The apparatus of clause 14, where the apparatus is configured to be a hand-held device.

16. The apparatus of clause 14 or clause 15, where: the control means includes means for estimating a change in cross-sectional area of the artery based at least in part the ultrasonic receiver signals; and estimating the blood pressure is based in part on the change in the cross-sectional area of the artery.

17. The apparatus of clause 16, where: the control means includes means for estimating a change in a blood flow rate within the artery based on the ultrasonic receiver signals; and estimating the blood pressure is based in part on the change in the blood flow rate within the artery and the change in cross-sectional area of the artery.

18. A blood pressure estimation method, including: controlling, by a control system, an ultrasonic sensor system to transmit ultrasonic waves to a target object on an outer surface of an apparatus, the ultrasonic sensor system having M ultrasonic receiver elements in a single array; receiving, by the control system from the ultrasonic sensor system, ultrasonic receiver signals from each of a plurality of the M ultrasonic receiver elements in the single array, the ultrasonic receiver signals corresponding to ultrasonic waves reflected from the target object; detecting, by the control system, an artery within the target object based on the ultrasonic receiver signals; and estimating, by the control system, a blood pressure within the artery based on the ultrasonic receiver signals.

19. The method of clause 18, further including estimating, by the control system, a change in cross-sectional area of the artery based at least in part the ultrasonic receiver signals, where estimating the blood pressure is based in part on the change in the cross-sectional area of the artery.

20. The method of clause 19, further including estimating, by the control system, a change in a blood flow rate within the artery based on the ultrasonic receiver signals, where estimating the blood pressure is based in part on the change in the blood flow rate within the artery.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

What is claimed is:

1. An apparatus, comprising:
an ultrasonic sensor system including a single array of M ultrasonic receiver elements; and
a control system configured to:
control the ultrasonic sensor system to transmit ultrasonic waves to a target object on an outer surface of the apparatus;
receive, from the ultrasonic sensor system, ultrasonic receiver signals from each of a plurality of the M ultrasonic receiver elements in the single array, the ultrasonic receiver signals corresponding to ultrasonic waves reflected from the target object;
detect an artery within the target object based on the ultrasonic receiver signals; and
estimate a blood pressure within the artery based on the ultrasonic receiver signals.

2. The apparatus of claim 1, wherein the apparatus is configured to be worn on a human wrist and wherein the artery is a radial artery.

3. The apparatus of claim 1, wherein the control system is further configured to apply a receiver-side beamforming process to the ultrasonic receiver signals, to produce a beamformed ultrasonic receiver image.

4. The apparatus of claim 3, wherein:
the control system is further configured to estimate a change in cross-sectional area of the artery based at least in part on the beamformed ultrasonic receiver image; and
estimating the blood pressure is based in part on the change in the cross-sectional area of the artery.

5. The apparatus of claim 4, wherein:
the control system is further configured to estimate a change in a blood flow rate within the artery based on the ultrasonic receiver signals; and
estimating the blood pressure is based in part on the change in the blood flow rate within the artery and the change in cross-sectional area of the artery.

6. The apparatus of claim 5, wherein estimating the blood pressure is based in part on a derivative of the blood flow rate (Q) within the artery with respect to the cross-sectional area (A) of the artery (dQ/dA).

7. The apparatus of claim 6, wherein the control system is configured to estimate a pulse wave velocity based on the derivative dQ/dA.

8. The apparatus of claim 5, wherein the control system is configured to estimate the change in the blood flow rate within the artery based at least in part on speckle decorrelation-based blood flow measurements or a Doppler-based method.

9. The apparatus of claim 5, further comprising a magnetic sensor system, wherein:
the control system is further configured to estimate a change in a blood flow rate within the artery based on magnetic sensor signals from the magnetic sensor system; and
estimating the blood pressure is based in part on the change in the blood flow rate within the artery and the change in cross-sectional area of the artery.

10. The apparatus of claim 5, further comprising an optical sensor system, wherein:
the control system is further configured to estimate a change in a blood flow rate within the artery based on optical sensor signals from the optical sensor system; and
estimating the blood pressure is based in part on the change in the blood flow rate within the artery and the change in cross-sectional area of the artery.

11. The apparatus of claim 1, wherein the ultrasonic sensor system is configured to provide ultrasonic receiver signals to the control system at a frame rate in a range from 1 KHz to 3 KHz.

12. The apparatus of claim 1, wherein the ultrasonic receiver signals include frequencies in a range from 10 MHz to 25 MHz.

13. The apparatus of claim 1, wherein the single array of M ultrasonic receiver elements is linearly arranged.

14. An apparatus, comprising:
an ultrasonic sensor system including a single array of M ultrasonic receiver elements; and
control system means for:
controlling the ultrasonic sensor system to transmit ultrasonic waves to a target object on an outer surface of the apparatus;
receiving, from the ultrasonic sensor system, ultrasonic receiver signals from each of a plurality of the M ultrasonic receiver elements in the single array, the ultrasonic receiver signals corresponding to ultrasonic waves reflected from the target object;
detecting an artery within the target object based on the ultrasonic receiver signals; and
estimating a blood pressure within the artery based on the ultrasonic receiver signals.

15. The apparatus of claim 14, wherein the apparatus is configured to be a hand-held device.

16. The apparatus of claim 14, wherein:
the control means comprises means for estimating a change in cross-sectional area of the artery based at least in part the ultrasonic receiver signals; and
estimating the blood pressure is based in part on the change in the cross-sectional area of the artery.

17. The apparatus of claim 16, wherein:
the control means comprises means for estimating a change in a blood flow rate within the artery based on the ultrasonic receiver signals; and
estimating the blood pressure is based in part on the change in the blood flow rate within the artery and the change in cross-sectional area of the artery.

18. A blood pressure estimation method, comprising:
controlling, by a control system, an ultrasonic sensor system to transmit ultrasonic waves to a target object on an outer surface of an apparatus, the ultrasonic sensor system having M ultrasonic receiver elements in a single array;
receiving, by the control system from the ultrasonic sensor system, ultrasonic receiver signals from each of a plurality of the M ultrasonic receiver elements in the single array, the ultrasonic receiver signals corresponding to ultrasonic waves reflected from the target object;
detecting, by the control system, an artery within the target object based on the ultrasonic receiver signals; and
estimating, by the control system, a blood pressure within the artery based on the ultrasonic receiver signals.

19. The method of claim 18, further comprising estimating, by the control system, a change in cross-sectional area of the artery based at least in part the ultrasonic receiver signals, wherein estimating the blood pressure is based in part on the change in the cross-sectional area of the artery.

20. The method of claim 19, further comprising estimating, by the control system, a change in a blood flow rate within the artery based on the ultrasonic receiver signals, wherein estimating the blood pressure is based in part on the change in the blood flow rate within the artery.
